(19) United States
(12) Patent Application Publication
(10) Pub. No.: US 2018/0289421 A1
(43) Pub. Date: Oct. 11, 2018

(54) SYSTEMS AND METHODS FOR TREATING NAIL FUNGUS

(71) Applicant: Terimber Corporation, San Jose, CA (US)

(72) Inventor: Iouri Poutivski, San Jose, CA (US)

(21) Appl. No.: 15/480,281

(22) Filed: Apr. 5, 2017

(51) Int. Cl. A61B 18/20; A61N 5/06; A61K 41/00; A61K 47/22; A61K 47/20

(52) U.S. Cl. CPC A61B 18/203; A61N 5/0624; A61B 2018/00982; A61K 47/22; A61K 47/20; A61K 41/0052

(57) ABSTRACT

Methods for treating dermal infection in an appendage of a mammal can include removing, such as by grinding off, a first nail portion of an appendage a mammal to expose a second nail portion of the appendage. A laser absorbing compound can be applied to the second nail portion of the appendage. The second nail portion can be irradiated with a laser of a treatment system to selectively heat the laser absorbing compound. Methods can also include applying a system for treating dermal fungal infection. A system can include a laser, an imaging unit for obtaining infection image data, and/or an alignment stage for receiving an infected portion of a mammal including the dermal fungal infection and aligning the infected portion of the mammal with the laser. A system can also include a control unit for controlling relative movement between the laser and the alignment stage.

### BACKGROUND

Up to 10% of adults in Western countries have a fungal infection of the nails. The percentage jumps to over 20% for adults who are age 60 or older. Types of treatment protocols vary widely and some carry associated risks. For example, oral treatments typically have an in vivo efficiency around 80% but many pose the risk of severe liver damage. In such treatments, a curing compound is delivered to a treatment site through blood flows. The concentration of the compound at the treatment site increases to a level where infecting funguses are affected.

Another type of treatment is topical treatment. Topical treatment is most effective for infections occurring at nail edges and is relatively ineffective for severely infected nails. Nail structure, and especially the keratin layer, has a high affinity for applied cure compounds and absorbs the compound far from the affected area. When the concentration of the compound at the infected location reaches a level sufficient to kill fungus, the nail often has grown out of and/or over the infected area. Finally, fungal infections are also treated surgically. However, a nail extraction to treat an infection can be very painful and does not have a guaranteed success rate.

A further type of treatment is laser treatment. During laser treatment, a laser is applied directly to a nail. However, heat required to kill funguses in such applications often exceeds a reasonable level to be applied to living organisms. As such, laser treatments have often resulted in substantial tissue damage or have not heated a fungus enough to substantially eradicate it.

### SUMMARY

Systems and methods for treating dermal infection in an appendage of a mammal are provided herein. The methods include applying a laser absorbing compound to a nail portion of the appendage and irradiating the nail portion with a laser of a treatment system to selectively heat the laser absorbing compound and treat the dermal infection.

In various embodiments, the subject systems include a laser and an alignment stage for receiving an infected portion of a mammal including the dermal fungal infection and aligning the infected portion of the mammal with the laser. Also, in some aspects, the laser and the alignment stage are operatively connected and configured to move relative to one another along an irradiating pattern, for example, a continuous irradiating pattern and/or a periodic irradiating pattern, to irradiate the infected portion of the mammal. In some versions, the systems also include one or more imaging unit for obtaining infection image data. In some versions, the infection image data can include luminescence detected from the infection. The subject systems can also include a tissue removing device operatively coupled to the control unit such that the control unit moves the tissue removing device with respect to the appendage to remove a nail portion of the infected portion of the mammal.

A laser can include a diode, a cooler, a lens, and/or an optical fiber, or any combination thereof. The laser can, in some embodiments include a diode configured to emit radiation having a wavelength of 850 nm or less, such as 810 nm or less, such as 800 nm or less. The laser can also include a first diode configured to emit radiation having a wavelength of 850 nm or less and/or a second diode configured to emit radiation detectable by an un-assisted human eye. Also, a lens of a laser can be cylindrical and/or have a cross-sectional diameter of 2.5 cm or less.

According to some aspects, the system includes a movement unit for moving at least a portion of the laser. In various embodiments, the system includes a movement unit for moving at least a portion of the alignment stage. A movement unit can be configured for moving the infected portion of the mammal in a first dimension and a second dimension perpendicular to the first dimension.

In some embodiments, the systems include a control unit for controlling the relative movement between the laser and the alignment stage and/or the intensity, pattern or frequency or irradiation. A control unit can be configured for controlling relative movement between the laser and the alignment stage along an irradiating pattern and/or irradiating an infected portion of the mammal based on obtained infection image data.

The systems can also include a power source operatively connected to the laser, the alignment stage, or both the laser and the alignment stage. The power source can also be operatively connected to the control unit.

Methods of treating a dermal fungal infection such as onychomycosis, in an appendage of a mammal are also disclosed herein. Such methods can include removing a first nail portion of an appendage a mammal including the dermal fungal infection to expose a second nail portion of the appendage; applying a laser absorbing compound such as indocyanine green (ICG) and/or a solvent such as dimethyl sulfoxide (DMSO) to the second nail portion of the appendage; and/or irradiating the second nail portion with a laser of a treatment system to selectively heat the laser absorbing compound. Such heating can kill tissue so that it can be removed to expose a deeper layer. Such heating can also kill dermal fungus associated with the dermal fungal infection. In various aspects, the laser absorbing compound is selectively heated to 80° C. or more.

According to some versions, the methods include stably associating the appendage of the mammal with an alignment stage of the treatment system. The methods can also include moving at least a portion of the alignment stage to align in treatment proximity the second nail portion and the laser.

Various aspects also include aligning in treatment proximity the second nail portion and the laser using radiation emitted by the laser as a visual alignment guide. Also, in some versions of the methods, irradiating the second nail portion includes continuously irradiating the second nail portion with the laser while continuously moving the second nail portion relative to the laser. In some aspects, irradiating the second nail portion includes periodically irradiating the second nail portion with the laser while continuously moving the second nail portion relative to the laser. Furthermore, in some versions, irradiating the second nail portion includes periodically irradiating the second nail portion with the laser and periodically moving the second nail portion relative to the laser.

In some aspects, the methods include removing one or more portions of the second nail portion. Removing a portion of the second nail portion can include contacting the portion of the second nail portion with a tissue removing device. Also, in some versions, the methods can further include inputting a set of instructions to a control unit of the treatment system operatively connected to a tissue removing device, wherein upon receiving the set of instructions, the control unit moves the tissue removing device with respect to the appendage to remove the portion of the second nail portion.

In some versions of the methods, removing a nail portion, e.g., a first and/or second nail portion, includes contacting the nail portion with a tissue removing device. The methods can also include administering an anesthetic compound to the second nail portion of the appendage. An appendage can, in various aspects, be a finger or a toe. In some aspects, the methods include removing a portion of the second nail portion of the appendage. Also, the subject methods can include administering an anti-infectious compound to the second nail portion of the appendage.

In various aspects, the methods further include repeating, e.g., repeating one or more times, e.g., two or more, five or more or ten or more or ten or fewer, five or fewer, or two or fewer, steps of applying a laser absorbing compound to the second nail portion of the appendage, irradiating the second nail portion with a laser of a treatment system to selectively heat the laser absorbing compound, and removing a portion of the second nail portion of the appendage. The steps can be repeated at a frequency of once every four weeks or less, once every two weeks or less, once per week or less, or once per day or less. In some versions, the methods further include repeatedly administering an anti-infectious compound to the second nail portion of the appendage at a frequency of once per day or more. The methods can also include increasing an amount of laser radiation delivered each time the second nail portion is irradiated with the laser.

Aspects of the methods also include inputting a set of instructions into a control unit of the treatment system, wherein upon receiving the set of instructions, the control unit moves the second nail portion in a continuous irradiation pattern or a periodic irradiation pattern with respect to the laser.

The disclosed subject matter also includes kits. The kits can include a treatment system including a laser and/or an alignment stage. The alignment stage can be configured for receiving an infected portion of a mammal including the dermal fungal infection and aligning the infected portion of the mammal with the laser. Also, the laser and the alignment stage are, in some aspects, operatively connected and configured to move relative to one another along an irradiating pattern to irradiate the infected portion of the mammal. The kits can also include a radiation absorbing compound, such as a laser absorbing compound, such as ICG. Such a compound can include a solvent, such as DMSO. Radiation absorbing compounds according to embodiments of the subject kits can also have a peak spectral absorption of 850 nm or less, such as 820 nm or less, such as 810 nm or less, such as 800 nm or less. The subject kits can also include a tissue removing device for removing a nail portion of the infected portion of the mammal. In some versions of the kits, a tissue removing device is operatively coupled to the control unit so that the control unit controls the removal of the nail portion of the infected portion of the mammal.

### BRIEF DESCRIPTION OF THE DRAWINGS

These and other aspects and features of the present invention will become apparent to those ordinarily skilled in the art upon review of the following description of specific embodiments of the invention in conjunction with the accompanying figures, wherein:

FIGS. 1A-1D provide diagrams representing a method of treating an infection according to embodiments of the present disclosure.

FIG. 2 provides a schematic illustration of a system in accordance with embodiments of the subject disclosure.

FIG. 3 provides a schematic illustration of a periodic irradiating pattern according to the subject disclosure.

FIG. 4 provides a schematic illustration of a continuous irradiating pattern according to the subject disclosure.

### DETAILED DESCRIPTION

Systems and methods for treating dermal infection in an appendage of a mammal are provided herein. The methods include applying a laser absorbing compound to a nail portion of the appendage and irradiating the nail portion with a laser of a treatment system to selectively heat the laser absorbing compound and treat the dermal infection.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Additionally, certain embodiments of the disclosed devices and/or associated methods can be represented by drawings which may be included in this application. Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Systems

Systems and devices for treating infections including dermal infections in a subject are included in the subject disclosure. Systems according to the subject embodiments include a laser and an alignment stage operatively connected and configured to move relative to one another along an irradiating pattern to irradiate an infected portion of the subject.

As used herein, the phrases “operatively connected” “operatively coupled” and “operably coupled” mean connected in a specific way that allows the disclosed methods to be carried out and/or systems to operate effectively in the manner described herein. For example, one aspect, such as a laser, which is operatively connected to a second aspect, such as an alignment stage, may be fixedly connected and/or removably connected, and/or electrically connected such that that electrical voltage may exist between and/or current or be transmitted from one connected aspect to another. One aspect operatively connected to another can also be reversibly attachable such that the two aspects can be physically coupled and de-coupled repeatedly.

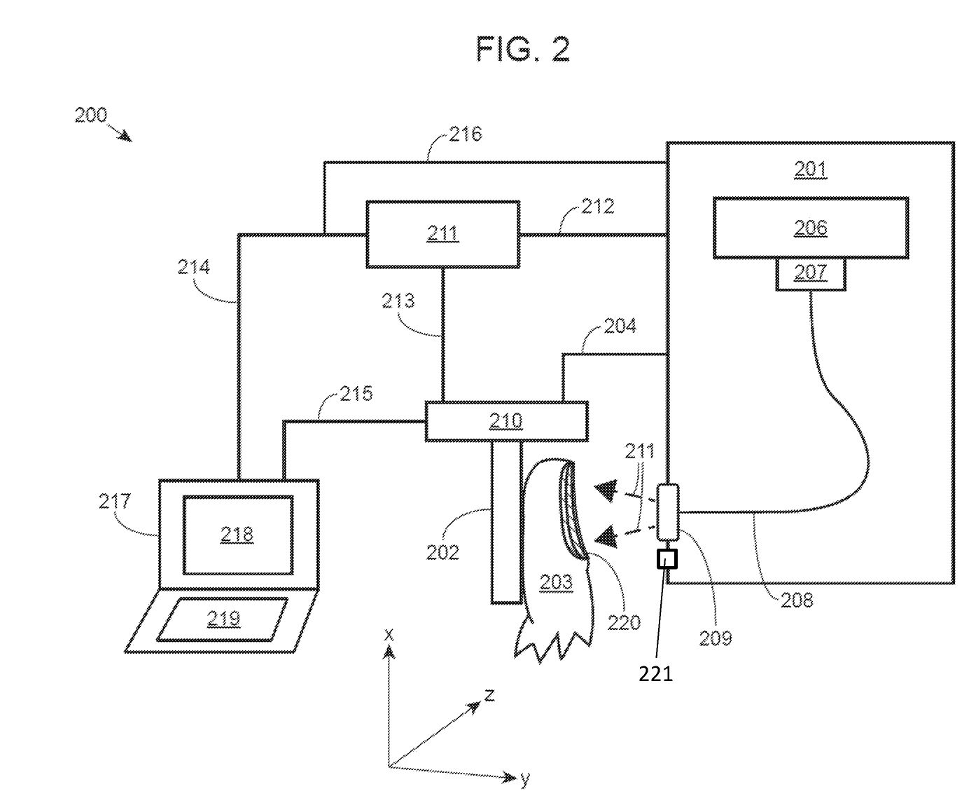

FIG. 2 provides a schematic illustration of a system in accordance with an embodiment of the disclosed subject matter. As is provided in FIG. 2, a system 200 includes a laser 201 and an alignment stage 202 for receiving infected portion of a mammal including the dermal fungal infection and aligning the infected portion of the mammal 203 including nail portion 220 with the laser 201. The laser 201 and the alignment stage 202 can be operatively connected as illustrated by operative connection 204. The laser 201 and the alignment stage 202 can also be configured to move relative to one another along an irradiating pattern to irradiate, such as with radiation 205, for example, laser radiation, the infected portion of the mammal 203.

In various aspects, the laser 201 includes any one or combination of a diode 207, a cooler 206 for cooling one or more aspects of the laser such as the diode 207, a lens, 209, and/or an optical fiber 208 for transmitting radiation from the diode 207 to the lens 209.

In some embodiments, the alignment stage 202 is operatively connected to a movement unit 210 for moving at least a portion of the alignment stage and/or the infected portion of the mammal 203, such as moving the at least a portion of the alignment stage 202 in one, two, or three dimensions as indicated by the axes x, y, and z in FIG. 2. In some embodiments, the movement unit 210 is configured for moving at least a portion of the alignment stage and/or the infected portion of the mammal 203, in only two dimensions. The movement unit 210 can be configured for moving the infected portion of the mammal 203 in a first dimension and a second dimension perpendicular to the first dimension. Furthermore, any one or combination of the components of the described systems can be connected to one another via operative connections, e.g., 204, 212, 213, 214, 215, 216.

Systems as provided herein can also include a power source 211 operatively connected, for example, via operative connections 212, 213, to the laser 201 and/or the alignment stage 202. A power source 211 can also be operatively connected to any one or combination of other system components described herein. Also, in some versions, the subject systems include an imaging unit 221, such as a camera, for obtaining image data. Such a camera can be operatively connected to the laser, control unit and/or alignment stage.

By “power source”, as used herein, is meant a device that supplies electric power to an electrical load. As such, in some aspects, power sources may include, for example, one or more battery, direct current (DC) power supply, alternating current (AC) power supply, linear regulated power supply, switched-mode power supply, programmable power supply, uninterruptible power supply, high-voltage power supply and/or a voltage multiplier. The amount of power, current and/or voltage capable of being provided by a power supply may, for example, be equivalent to that of or an electronic media player or communication device such as a computer, or one or more components thereof, a television, and/or a telephone.

Embodiments of power sources include power sources configured to turn on to provide electrical power to another component and/or turn off to stop providing electrical power to another component. Such power sources may be configured to be turned on and/or off, for example, by operation of a switch, button, timer or other component operatively connected to or included in the power source.

A power source may, in certain aspects, be operatively connected to one or more components of the disclosed systems. As such, embodiments of power sources include electrical connections from a power source to components of the disclosed systems. Such electrical connections may include one or more lengths of electrically conductive material such as contacts and/or wires.

Embodiments of power sources include a wide variety of shapes and sizes including, for example, all possible combinations of the shapes and sizes of various components described herein. One or more power sources may, in certain aspects, be operatively, e.g., adhesively, snapedly, hingedly or otherwise, connected to one or more components of the disclosed systems. In certain aspects, all or portion of the power source may be on the interior and/or the exterior of another component of the disclosed systems such as a housing.

The subject systems can also include a control unit 217 operatively connected to any one or combination of other system components described herein. A control unit can be configured for controlling the relative movement between the laser and the alignment stage by for example, controlling movement of the laser and/or movement of one or more portions of the alignment stage. A control unit 217 can also include an input 218 for receiving instructions to be executed by the control unit and/or a display 219 for providing an output to a user of the control unit.

A control unit 217, in various embodiments, can be a computer. Aspects of the subject computers may include one or more components, which are operatively coupled to each other, such as physically, electrically and/or communicatively. A computer may include one or more controllers, e.g., a processor, for receiving an input and/or generating an output. For example, a controller may be configured for storing and/or executing instructions presented to it and/or may be operatively coupleable to a power source. A computer may also include one or more display for displaying one or more output. A display may be configured for displaying one or more forms of digital media, such as content on a webpage. An output may be presented by the computer in response to an input of instructions to the computer, such as by operatively coupling an instruction carrying device, such as a flash drive, to the controller. The subject devices may also include a graphical user interface. A computer may also be in remote communication with, e.g., wired or wireless communication with, one or more instruction carrying device, such as one or more cellular telephones. Also, where devices include one or more computers, the computers may each include one or more input device, such as a mouse and/or keyboard, and/or controller and/or display, such as a presence-sensitive screen, and/or a voice responsive system, and/or a camera, and/or a video recorder, and/or a microphone, and/or a GPS module, or any other type of device for detecting a command from a user or sensing the environment. In some aspects, a presence-sensitive screen includes a touch-sensitive screen.

Various components and/or parties of the present disclosure may communicate, such as communicate to and/or from the computer, over a network including electronic devices connected either physically or wirelessly, wherein digital information is transmitted from one device to another. Such devices, such as end-user devices and/or servers, may include, but are not limited to: a desktop computer, a laptop computer, a handheld device or PDA, a cellular telephone, a set top box, an Internet appliance, an Internet TV system, a mobile device or tablet, or systems equivalent thereto. Exemplary networks include a Local Area Network, a Wide Area Network, an organizational intranet, the Internet, or networks equivalent thereto.

In various embodiments, the present disclosure is directed to one or more computer systems capable of carrying out the functionality and/or aspects of the methods described herein. A computer system can include one or more processors. A processor is connected to a communication infrastructure (e.g., a communications bus, cross-over bar, or network). Computer system can include a display interface that forwards graphics, text, and other data from the communication infrastructure (or from a frame buffer not shown) for display on a local or remote display unit.

Computer system also can include a main memory, such as random access memory (RAM), and may also include a secondary memory. The secondary memory may include, for example, a hard disk drive and/or a removable storage drive, representing a floppy disk drive, a magnetic tape drive, an optical disk drive, flash memory device, etc.

According to some aspects of the subject devices, secondary memory may include other similar devices for allowing computer programs or other instructions to be loaded into computer system. Such devices may include, for example, a removable storage unit and an interface. Examples of such may include a program cartridge and cartridge interface (such as that found in video game devices), a removable memory chip (such as an erasable programmable read only memory (EPROM), or programmable read only memory (PROM)) and associated socket, and other removable storage units and interfaces, which allow computer software, instructions, and/or data to be transferred from the removable storage unit to computer system.

In some embodiments, a communications interface may be included in computer system. Communications interface allows computer software, instructions, and/or data to be transferred between computer system and external devices. Examples of communications interface may include a modem, a network interface (such as an Ethernet card), a communications port, a Personal Computer Memory Card International Association (PCMCIA) slot and card, etc. Software and data transferred via communications interface are in the form of signals which may be electronic, electromagnetic, optical or other signals capable of being received by communications interface. These signals are provided to communications interface via a communications path such as a channel. This channel carries signals and may be implemented using wire or cable, fiber optics, a telephone line, a cellular link, a radio frequency (RF) link, a wireless communication link, and/or other communications channels.

The terms “computer program medium,” “computer-readable storage medium,” and “computer usable medium” are used herein to generally refer to media such as removable storage drive, removable storage units, data transmitted via communications interface, and/or a hard disk installed in hard disk drive. These computer program products provide computer software, instructions, and/or data to computer system. These computer program products also serve to transform a general purpose computer into a special purpose computer programmed to perform particular functions, pursuant to instructions from the computer program products/software. Embodiments of the subject disclosure are directed to such computer program products.

Some embodiments of computer programs (also referred to as computer control logic) are stored in main memory and/or secondary memory. Computer programs may also be received via communications interface. Such computer programs, when executed, enable the computer system to perform the features of the present disclosure, as discussed herein. In particular, the computer programs, when executed, enable the processor to perform the features of the presented methods. Accordingly, such computer programs represent controllers of the computer system. Where appropriate, the processor, associated components, and equivalent systems and sub-systems thus serve as “means for” performing selected operations and functions. Such “means for” performing selected operations and functions also serve to transform a general purpose computer into a special purpose computer programmed to perform the selected functions and operations.

In embodiments where aspects of the subject disclosure are implemented using software, the software may be stored in a computer program product and loaded into computer system using removable storage drive, interface, hard drive, communications interface, or equivalents thereof. The control logic (software), when executed by the processor, causes the processor to perform the methods and functions described herein.

The computer systems described herein may also include one or more power sources to provide power to the computer system. Non-limiting examples of power sources include single-use power sources, rechargeable power sources, and/or power sources developed from nickel-cadmium, lithium-ion, or other suitable material, such as batteries.

Output devices may also be included in a computer system of the present disclosure. Output devices are configured to provide output to a user using, for example, tactile, audio, and/or video stimuli. Output devices may include a display screen (part of the presence-sensitive screen), a sound card, a video graphics adapter card, or any other type of device for converting a signal into an appropriate form understandable to humans or machines. Additional examples of output devices include a speaker, a cathode ray tube (CRT) monitor, a liquid crystal display (LCD), or any other type of device that can generate intelligible output to a user. In some embodiments, a device may act as both an input device and an output device.

In some aspects of the disclosure, there is provided an article of manufacture including: a machine-readable medium having machine-readable instructions stored thereon, the instructions including: instructions for providing a medical professional and/or a patient with information regarding one or more aspects of the methods described herein; instructions for providing the user with a plurality of customization options for the display of one or more aspects of the methods selected by the user for display via the digital medium; instructions for receiving one or more user selections for the customization options; and instructions for displaying via the digital medium the information regarding one or more aspects of the methods selected by the user for display via the digital medium in accordance with the one or more user selections for the customization options.

As noted above, embodiments of the subject systems include a laser. The laser can include a diode, a cooler, a lens, an optical fiber, or any combination thereof. In various aspects, the laser includes a diode, such as a first diode. The diode can be configured to emit laser radiation having a wavelength of 2000 nm or less, such as 1500 nm or less, such as 1000 nm or less, such as 850 nm or less, such as 830 nm or less, such as 815 nm or less, such as 810 nm or less, such as 805 nm or less, such as 800 nm or less, such as 780 nm or less. A diode can also be configured to emit laser radiation having a wavelength of in an inclusive range of for example, 600 nm to 2000 nm, such as 600 nm to 1000 nm, such as 700 nm to 900 am, such as 750 nm to 850 nm, such as 780 nm to 810 nm.

Also, lasers according to various aspects are short pulse lasers or long pulse lasers. Such lasers may be solid state or non-solid state and can include neodymium-doped yttrium aluminum garnet (Nd:YAG) lasers. Lasers according to the various embodiments can also include Q-switched lasers. Also, lasers which may be applied include dual wavelength diode lasers, and fractional CO₂ lasers.

In some aspects, the laser includes a plurality of diodes, such as a first diode and a second diode. Each of the diodes may be configured to emit radiation at the same wavelength or at different wavelengths. In various embodiments, the first diode is configured to emit radiation at a first wavelength or within a first range of wavelengths the second diode is configured to emit radiation at a second wavelength or within a second wavelength range, wherein the second wavelength and the second wavelength range are different than the first wavelength and the first wavelength range. In various embodiments, the first diode is configured to emit radiation which is not optically detectable by an un-assisted human eye. The term “human,” as used herein, can include human users or subjects of both genders and at any stage of development, such as fetal, neonates, infant, juvenile, adolescent, adult, where in certain embodiments the human subject or user is a juvenile, adolescent or adult. Also, an un-assisted human eye refers to a human eye that is not enhanced by one or more devices which enhance or modify visual ability. Such devices might include a camera, optical magnifier, microscope, or optimized, such as filtered, e.g., polarized, glasses or contacts, etc.

Also, in various embodiments, the second diode is configured to emit radiation which is optically detectable by an un-assisted human eye. In some aspects, the second diode is configured to emit radiation, the optical detection of which is applied for alignment of radiation emitted by the first diode with an infected portion of an appendage of a subject. In such embodiments, the second diode is part of an alignment element of the laser which can be used to adjust the spatial position such as the distance, of a lens of the laser with respect to an infected portion of a mammal on an alignment stage. In various embodiments, the second diode is not a laser diode.

In various embodiments, a laser may include one or more lenses, such as a first lens configured to focus radiation emitted from a first diode and/or a second lens configured to focus radiation emitted from a second diode. Lenses according to the subject disclosure can have a cross-sectional diameter of 10 cm or less, such as 5 cm or less, such as 3 cm or less, such as 2.5 cm or less, such as 2 cm or less, such as 1.5 cm or less, such as 1 cm or less, such as 0.5 cm or less. Lenses according to the disclosure can also have a cylindrical shape and/or be composed of glass or a light-transmissive polymeric material such as plastic.

In various embodiments, the laser is configured to heat an irradiated aspect, such as a laser absorbing compound and/or tissues contacting or adjacent to the compound from an initial temperature, such as room-temperature and/or 20° C. to 80° C. or more, such as 85° C., such as 90° C. or more. In some versions, the laser is configured to heat an irradiated aspect 10° C. or more, 20° C. or more, 30° C. or more, 40° C. or more, 50° C. or more, 60° C. or more, 70° C. or more, or 80° C. or more.

In some versions, heat capacity (H) of an irradiated aspect, such as an appendage, or a component thereof, such as water, is, for example, H=4.1855 J/g/K and the density (D) is D=1 g/cm̂3. Thus the required energy (E) for volume cm̂3 is E=H*D*(80−20)=250 J. The required energy (E) to heat such an aspect, for example, from 20° C. to 80° C., in terms of energy for volume cm̂3, can range from 100 J to 500 J, such as 200 J to 300 J, such as 240 J to 260 J.

For an area with smaller dimensions, e.g., 1 mm by 1 mm and depth 1 mm or 1 mm̂3, =10̂-3 cm̂3 then E=250 J*10̂-3=250 mJ and for 200 microns×200 microns×1 mm microns 250 mJ/25=10 mJ. The required energy (E) to heat such an aspect, for example, from 20° C. to 80° C., in terms of energy for volume cm̂3, can range from 1 mJ to 100 mJ, such as 5 mJ to 50 mJ, such as 10 mJ to 20 mJ.

Accordingly, power of laser irradiation applied with the laser to heat such an aspect, for example, from 20° C. to 80° C., at the above estimation of the pulse duration 10 ms, the CW power of the laser is P=10 mJ/10 ms=1 W. The 10-ms pulse duration is derived from two values. The first value is the typical speed of the mechanical stages, e.g., 1 inch/second, as such values are described further below. The second value is the diameter of the focused laser beam, e.g., ˜200 microns, as described for example above. Power of laser irradiation applied with the laser to heat such an aspect, for example, from 20° C. to 80° C. can range, for example, from 0.1 W to 10 W, such as 0.5 W to 5 W, such as 1 W to 2 W. An exemplary laser which can be applied in accordance with the subject embodiments is the QSP-808-4 by QPhotonics®. The QSP-808-4 has P=4 W.

Embodiments of the subject systems also include an alignment stage. An alignment stage can include a first portion and a second portion. Each portion can be configured to move relative to the other, for example, such that the first portion moves in two dimensions, such as only in two dimensions, relative to the second portion. The first portion can be configured to slide along a length of the second portion. The first portion can be a receiving portion for receiving an infected portion of a mammal. The second portion can be an anchored portion which is anchored and stationary relative to the first portion while the first portion moves. Each of the first and second portions can be planar.

In various embodiments, alignment stages are configured for aligning the infected portion of the mammal with the laser. Aligning the infected portion of the mammal with the laser includes either retaining a portion of the mammal in a stationary position or moving, such as moving toward and/or away along an axis and/or in one or more directions perpendicular to the axis, and then retaining the portion of the mammal in a stationary position so that the laser can irradiate the infected portion of the mammal and/or a laser-absorbing compound thereon as described herein.

Aligning the infected portion of the mammal with the laser can include positioning the portion of the mammal an effective distance away from the laser lens by moving the infected portion toward and/or away from the lens along an irradiation axis. An irradiation axis can be perpendicular to the surface of an infected portion of a mammal and/or a system aspect such as a surface of an alignment stage or a portion thereof, such as a first or second portion, and/or a laser portion, such as a lens. The effective distance can extend from the surface of the infected portion of the mammal to the surface of the laser lens. The effective distance can be, for example, 500 cm or less, 200, cm or less, 100 cm or less, 10 cm or less, 9 cm or less, 8 cm or less, 7 cm or less, 6 cm or less, such as 5 cm or less, such as 4 cm or less, such as 3 cm or less, such as 2 cm or less, such as 1 cm or less, such as 5 mm or less, such as 1 mm or less, or any other length or other dimensions that are compatible with the functionality of the system. The effective distance can be, for example, 500 cm or more, 200, cm or more, 100 cm or more, 10 cm or more, 9 cm or more, 8 cm or more, 7 cm or more, 6 cm or more, such as 5 cm or more, such as 4 cm or more, such as 3 cm or more, such as 2 cm or more, such as 1 cm or more, such as 5 mm or more, such as 1 mm or more, or any other length or other dimensions that are compatible with the functionality of the system. The effective distance can also range from, for example, 1 mm to 500 cm, such as 1 mm to 200 cm, such as 1 mm to 100 cm, such as 1 mm to 10 cm, such as from 1 mm to 5 cm, such as from 1 mm to 3 cm, such as from 1 mm to 1 cm, or any other length or other dimensions that are compatible with the functionality of the system.

Aligning the infected portion of the mammal with the laser can include positioning the portion of the mammal with respect to laser lens by moving the infected portion laterally from the lens in one or more directions perpendicular to the irradiation axis. The infected portion of the mammal can be moved laterally from the lens along a length equaling any of the distances provided above in association with the “effective distance.”

In various embodiments, the alignment stage is operatively connected to or includes a movement unit for moving at least a portion, such as a first and/or second portion, of the alignment stage. Also, in various embodiments, the laser is operatively connected to or includes a movement unit for moving at least a portion, such as a lens, of the laser. As noted above, aligning the infected portion of the mammal with the laser can include positioning the portion of the mammal an effective distance away from the laser lens by moving the infected portion toward and/or away from the lens along an irradiation axis or moving it laterally with respect to the lens in one or more directions perpendicular to the irradiation axis. Both can be performed by the movement unit operatively connected to the alignment stage. However, aligning the infected portion of the mammal with the laser can also include positioning the portion of the mammal an effective distance away from the laser lens by moving the lens toward and/or away from the infected portion along an irradiation axis or moving the lens laterally with respect to the infected portion in one or more directions perpendicular to the irradiation axis. Both can be performed by the movement unit operatively connected to the laser.

In various embodiments, movement units are configured for moving the infected portion of the mammal in a first dimension, such as a dimension perpendicular or parallel to an irradiation axis and/or a second dimension perpendicular to the first dimension. Also, in some embodiments, movement units are configured for moving the laser lens in a first dimension, e.g., a dimension perpendicular or parallel to an irradiation axis and/or a second dimension perpendicular to the first dimension.

In some embodiments, movement units of the subject devices have a one or more portions, such as one or more handle, such as a handle shaped as a rectangular plate, configured for manual actuation of the movement unit. In some embodiments, movement units as described herein are configured to rotate, such as rotate about an axis, such as a single axis, such as an axis of symmetry.

Embodiments of the disclosed systems also include movement units which are automatic movement units. Automatic movement units can include an automatic controller, such as an electronic controller, such as a computer controller, which can be a control unit as described herein, for controlling actuation of the movement units. Automatic movement units, in some embodiments, are not manually operated movement units and are configured to operate without an operator performing a manual actuation of the device. In various embodiments, automatic movement units may include an interface, such as an input, e.g., buttons and/or switches, for receiving instructions, such as instructions designating when and/or how a movement unit will actuate, from an operator. Automatic movement units may provide automatic actuation between, for example, first and second configurations, e.g., a first configuration and a second configuration at which a moved aspect is a distance away from the position it was in initially, one or a plurality of times. Such automatic actuation can be actuation in a first direction, such as a first linear or rotational direction, and/or a second direction multiple times and/or in varying degrees. Embodiments of automatic movement units can also provide actuation on a schedule set by a controller, such as a schedule that designates actuation or patterns of actuation that occur on regular or irregular intervals. Automatic movement units may also be configured to actuate based on an input from a separate source, such as one or more sensors, such as analyte and/or temperature sensors, or a timer of a system. Embodiments of automatic movement units can also include a display for producing an output providing information relating to actuation for an operator. Automatic movement units can include wireless capabilities, for example, for wireless activation and/or deactivation. Embodiments of automatic movement units can also be operated via a mobile device, such as a mobile telephone. Automatic movement units may also include one or more computer and/or power source, such as a battery, and/or can be operatively coupled to a power source. Furthermore, automatic movement units may include one or more propelling element, such as a motor, such as an electric motor, such as an electric step motor, to move the movement unit or a portion thereof in actuation and/or to drive actuation of the movement unit or portion thereof in one or more directions, e.g., a first direction, such as a rotational or linear direction, and/or a second direction opposite the first direction.

In various embodiments, alignment stages or portions thereof, such as one or more receiving portions, are configured for receiving an infected portion of a mammal and thereby stably retaining the portion of the mammal without substantial motion while it is irradiated according to the subject methods. As such, alignment stages or portions thereof may include one or more, such as a single and solitary, surface for stably contacting an appendage of a mammal or a portion thereof, e.g., a hand and/or foot and/or finger and/or toe. Such a surface may have an area sized to contact an appendage, such as a finger and/or toe, of a mammal. Such an area may be, for example, 1 cm² or less, 2 cm² or less, 5 cm² or less, 10 cm² or less, or 25 cm² or less.

An alignment stage or a portion thereof may be configured to hold and/or receive a portion of a mammal. In some versions, a portion of a mammal received by an alignment stage portion lies against a single receiving surface of the alignment stage portion. In some versions, a portion of a mammal received by an alignment stage portion, such as a hand and/or foot and/or finger and/or toe, lies partially or substantially within, such as between two or more portions, such as opposite interior portions of, or encapsulated within, the alignment stage portion.

Various embodiments of alignment stages or portions thereof, such as one or more receiving portions, include one or more openings (e.g., two, three, four, five openings) configured to receive a portion of a mammal, such as an infected appendage or a portion thereof, e.g., a hand and/or foot and/or finger and/or toe. Such an opening may separate an interior form an exterior of the alignment stage portion and may be shaped as any convenient shape, such as a circle, oval, square, rectangle, triangle, or any combination thereof. An opening of an alignment stage may also be sized to receive a portion of a mammal therethrough, such as a finger and/or a toe. An opening of an alignment stage may have, for example, an area of 1 cm² or less, 2 cm² or less, 5 cm² or less, 10 cm² or less, 25 cm² or less, 100 cm² or less, or 225 cm² or less. The opening of an alignment stage may be the only single opening from the interior to the exterior of the stage. If there is a plurality of openings on the element, the openings may be at a single first end rather than at a first and second opposite end. The subject methods, which are described further below, can include inserting a portion of an appendage of a subject, for example, a distal portion which is opposite a proximal portion an which includes an end of the appendage, into and/or through an opening of an alignment stage and/or stably associating a portion of an appendage of a subject, e.g., a distal portion, with a portion of an alignment stage.

Each of the first and second portions of an alignment stage, or other components described herein, such as a laser, control unit, power source, or a housing, or components thereof can include a sheet, such as a solid sheet, of one or more materials, for example, two materials, having a thin and/or planar shape. The components may include a top surface and a bottom surface each defining a plane parallel with the other and separated by a thickness. In some versions, protrusions extend from the top and/or bottom surface. In various embodiments, a component or a portion thereof, such as a first and/or second portion, is or includes a uniform layer of a single material. A body may also be composed of two or more, e.g., three, four, five, or more, etc. sheets laminated to one another.

A component or a portion thereof may, in some aspects, have a length, a width and a height, also referred to as a thickness. A component or a portion thereof may be shaped as a rectangular box with the width and length being substantially greater than the thickness. A length and/or width and/or thickness of a component or a portion thereof, e.g., a thickness between a first surface and a second surface opposite the first surface, may be 1 m or less, such as 0.5 m or less, such as 25 cm or less, such as 20 cm or less, such as 10 cm or less, such as 5 cm or less, such as 1 cm or less, such as 5 mm or less, 3 mm or less, 1 mm or less, or 0.5 mm or less. A length and/or width and/or thickness of a component or component portion may also range from 1 mm to 1 m, such as 1 mm to 50 cm, such as 1 mm to 40 cm, such as from 1 cm to 30 m, such as from 1 cm to 10 cm, such as from 1 cm to 5 cm, or from 1 mm to 5 cm, from 1 mm to 3 cm, from 1 mm to 1 cm or from 1 mm to 5 mm, each range inclusive.

Aspects of component or component portions may have an area defining a size or shape including a: circle, semi-circle, oval, rectangle, square, triangle, polygon, quadrilateral, or combination thereof. For example, in embodiments where the component or portion is shaped a rectangle, the length of the body is greater than the width. A component or portion may include one or more sheets of solid, uniform, integrated material, and in some versions, may not include any openings extending therethrough.

A component or component portion may have three edges, four edges, or more than four edges which define the area of the body. In various embodiments, the edges meet at corners, e.g., three, four, five, or ten or more corners. In some versions, a first edge of a layer is opposite a second edge of a layer and adjacent to a third and/or fourth edge of a layer. In such an embodiment, the third edge may be opposite a fourth edge and the fourth edge may be adjacent to the first and/or second edge. Also, in some versions, a component or component portion does not include one or more protrusions extending therefrom.

On some versions, the systems include one or more imaging unit such as a camera for obtaining infection image data. Infection image data is image data, such as recorded image data, e.g., a photo and/or video. In some aspects, the infection image data can include a digital photo and/or video, of a portion of a mammal, for example, a nail portion of a mammal and/or an infected portion of a mammal. Infection image data can also include detected radiation such as luminescence, such as luminescence produced by an infection. In some aspects, an infection can produce luminescence, absorbing a laser radiation of the particular wavelength. In some versions, a luminescence change is detected. Where appropriate, an amount of detected luminescence correlates to an amount of living fungus. Also, in some aspects, an absence of luminescence is detected when a fungus is killed or substantially killed. In addition, luminescence can be detected from a living fungus and the laser radiation can be applied to a luminescent area at a particular place or in a particular pattern based on the detected luminescence. An imaging unit can be operatively connected to a laser, alignment stage and/or control unit according to the subject embodiments.

A system or aspect thereof, such as a control unit, can also include a database including stored image data, e.g., infection image data or features thereof and associated data, for analyzing, such as by comparing with, infection image data obtained. Analysis data can include one or more characteristics or classifications, such as names, numbers, or other designations, etc., associated with a particular image aspect, e.g., brightness, color, shape and/or size of one or more aspects of an image. Furthermore, infection image data obtained can also be added to and/or stored in a database of a control unit for later communication to an external source, reference and/or analysis. The control unit can compare, such as automatically compare, stored analysis data with infection image data obtained to produce an irradiating pattern output and thereby control movement along the selected irradiating pattern. Also, irradiating pattern output can also be generated manually by evaluating one or more aspects of infection image data and/or analysis data associated therewith displayed on a display and/or entering one or more input into the control unit on the displayed data.

Additionally, a control unit can control relative movement between the laser and the alignment stage along an irradiating pattern based on obtained infection image data. A control unit can also control irradiation pattern, intensity and/or frequency of the infected portion of the mammal based on obtained infection image data.

The subject systems can also include one or more housings. A housing can be configured to enclose or substantially enclose one or more elements described herein, such as a laser, a control unit, an alignment stage and/or a power source. A plurality of housings can be included in a system. Housings can have any of the spatial characteristics described above.

System components or portions thereof which are described herein are composed of one or more materials, e.g., a plurality of materials, such as polymeric materials and/or metallic materials. In some embodiments, polymeric materials of the subject components, such as housings and/or insulation for electrodes forming operative connections, include, but are not limited to: glass fiber, polyimide, such as polyimide film, for example, Kapton® or Nomex®, and/or plastics, such as polytetrafluoroethene or polytetrafluoroethylene (PFTE), including expanded polytetrafluoroethylene (e-PFTE), polyester (Dacron®), nylon, polypropylene, polyethylene, high-density polyethylene (HDPE), polyurethane, polyexpoxide, phenol formaldehyde, etc. In some embodiments, metallic materials of the subject components, such as electrodes forming operative connections, include, but are not limited to conductive metals. Such metals can include copper, tin, silver, aluminum, bismuth, zinc, indium antimony, stainless steel, gold, titanium, tantalum, etc. and/or metal alloys.

In some embodiments, the laser and the alignment stage are configured to move relative to one another along an irradiating pattern to irradiate the infected portion of the mammal. When a portion of a mammal is irradiated, radiation, such as laser radiation, is emitted thereon and/or onto a substance on the mammal portion such as a laser-absorbing compound. Such irradiation can be sufficient to kill one or more causes of the infection such as a fungus. Also, movement between a laser and an alignment stage is performed by moving one of the elements with respect to the other in an irradiating pattern and can be driven by one or more, such as a plurality such as two, movement units. An irradiating pattern is a pattern on the surface of an infected portion of a mammal along which the infected portion is irradiated according to the subject methods. The irradiating pattern can be specific and pre-selected. The pattern can be optimized to maximize killing of an infecting aspect such as a fungus. Such optimization can be performed based on the stage in treatment and/or the type of the infecting aspect. An irradiating pattern can be a linear and/or curved pattern.

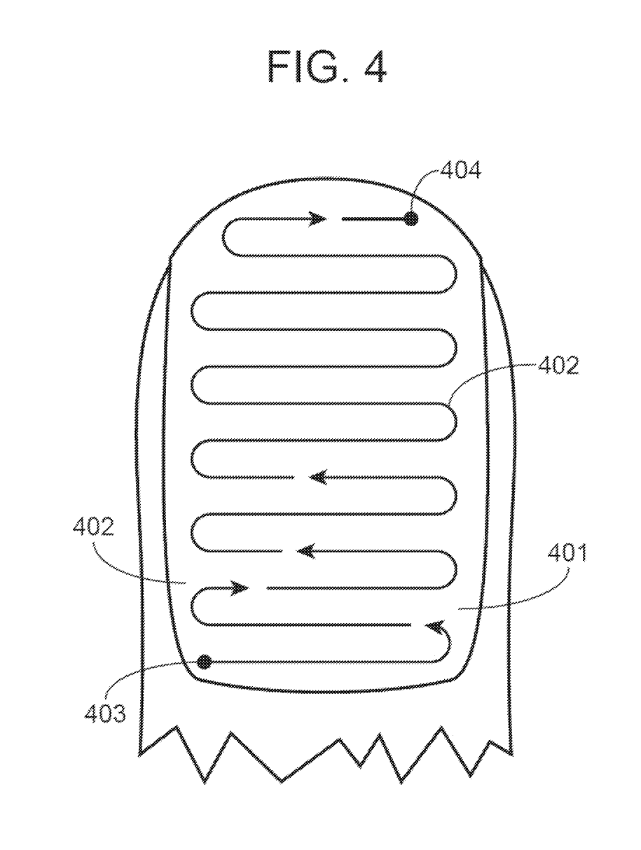

An irradiating pattern can be continuous or periodic. In a continuous irradiating pattern, the surface of the mammal is continuously irradiated. One schematic embodiment of a continuous irradiating pattern is shown, for example in FIG. 4. As is shown in FIG. 4, a nail portion of a mammal 401 is irradiated along the continuous irradiating pattern 402. As is provided in the figure, irradiation begins at a start point 403 and proceeds along the continuous irradiating pattern 402 to end point 404. In such a process, movement of the infected portion of the mammal with respect to the laser can be continuous or can be discrete. When movement is discrete, continuous irradiation occurs while relative movement is taking place and when relative movement is not taking place.

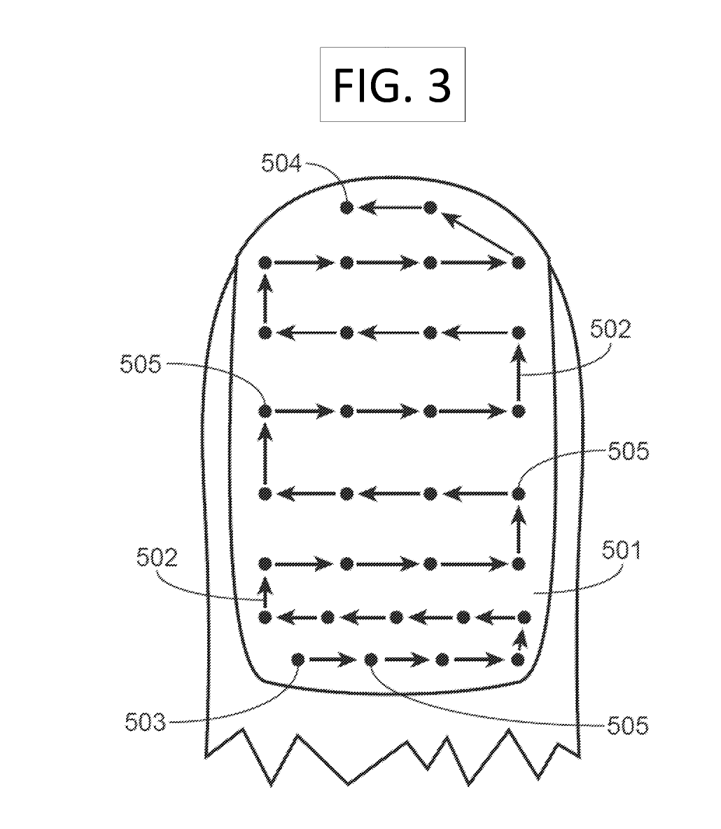

In a periodic irradiating pattern, the surface of the mammal is periodically irradiated. One schematic embodiment of a periodic irradiating pattern is shown, for example in FIG. 4. As is shown in FIG. 3, a nail portion of a mammal 501 is irradiated along the periodic irradiating pattern 502. The periodic irradiation pattern 502 proceeds along a series of irradiation areas 505 where irradiation occurs. The irradiation areas can be points on a surface or a mammal or can be linear. As is provided in the figure, irradiation begins at a start point 503 and proceeds along the periodic irradiating pattern 502 to end point 504.

In such a process, movement of the infected portion of the mammal with respect to the laser can be continuous or can be discrete. When movement is discrete, periodic irradiation occurs while relative movement is taking place and/or when relative movement is not taking place. In various embodiments, relative movement and irradiation alternate. In such aspects, there is no relative movement while irradiation occurs. Relative movement then occurs after irradiation followed by another period of irradiation.

Also, in a periodic irradiation pattern 502 there can be a distance 506, such as an average distance, between irradiation areas 505. Such a distance can be 5 cm or less, such as 4 cm or less, such as 3 cm or less, such as 2 cm or less, such as 1 cm or less, such as 5 mm or less, 4 mm or less, 3 mm or less, 1 mm or less, 1 mm or less, 0.7 mm or less, 0.5 mm or less, 0.3 mm or less, 0.2 mm or less, 0.1 mm or less, 0.01 mm or less, or 0.001 mm or less. Such a distance can also be 5 cm or more, such as 4 cm or more, such as 3 cm or more, such as 2 cm or more, such as 1 cm or more, such as 5 mm or more, 4 mm or more, 3 mm or more, 1 mm or more, 1 mm or more, 0.7 mm or more, 0.5 mm or more, 0.3 mm or more, 0.2 mm or more, 0.1 mm or more, 0.01 mm or more, or 0.001 mm or more. Furthermore, such as distance can range, for example, from 5 cm to 0.001 mm, such as 1 cm to 0.01 mm, such as 0.1 cm to 0.1 mm, or from 0.1 mm to 0.001 mm or 0.1 mm to 0.0001 mm.

Furthermore, an irradiating pattern can proceed along a path having any shape, for example, a circle, oval, square, rectangle, triangle, or any combination thereof. An irradiating pattern can, for example, be a repeating pattern wherein irradiation along the pattern is performed two or more times. An irradiating pattern can also be a reversible pattern wherein irradiation along the pattern is performed along the pattern in the first direction and then back along the pattern along a second and opposite direction. An irradiating pattern can proceed in a first direction along a first axis and then in a second direction along an axis perpendicular to the first. The pattern can then proceed in a third direction along an axis parallel with the first, wherein the third direction is opposite the first, and then in a fourth direction along an axis parallel with the second, wherein the fourth direction is the same as the second direction. Such a pattern can proceed in successive repeated steps until the entirety of second portion of the nail is irradiated.

Also, in some embodiments, an irradiating pattern extends over a surface area that is bigger than the surface area, e.g., nail portion, of the subject being treated. As such, an irradiating pattern can include areas in which a nail portion is irradiated and areas in which a nail portion is not irradiated. Furthermore, because, for example, laser radiation at around 800 nm is not substantially destructive and as such, is essentially harmless for human tissues including nail tissues, tissues of a subject in addition to a nail portion can also be included in an irradiating pattern. In addition to one or more nail portions, such areas can also be irradiated during a treatment as described herein. Also, radiation such as laser radiation at or around ˜800 nm, is not substantially absorbed by human tissue or is absorbed in an amount substantially less than as by laser absorbing compounds as described herein. As such, a laser irradiation pattern can be a solid area of irradiation having a shape such as a rectangle, square, oblong, and/or circular shape. Such a pattern can fully cover an area, e.g., a smaller area, to which a laser absorbing compound is applied as well as a larger area including tissues not having the compound applied. As such, the methods as provided below can include irradiating tissues to which a laser absorbing compound is applied as well as surrounding tissues to which the compound has not been applied. In such instances, applied laser radiation does not produce any noticeable hitting, e.g., destructive, effect on the tissue with no compound applied due to the near-infrared window in the biological tissue absorption spectrum.

### Methods

In various embodiments, the subject disclosure includes methods of treating an infection, such as a dermal fungal infection, in an appendage of a subject, such as a mammal. The methods can include removing a nail portion, such as a first nail portion, of an appendage a subject. After the first nail portion is removed, the methods can also include applying a laser absorbing compound to a second nail portion of the appendage. Thereafter, the second nail portion can be irradiated according to the subject methods with a laser of a treatment system. Such irradiation can include selectively heating the laser absorbing compound and thereby killing tissue, such as tissue of the second nail portion, and/or dermal fungus. Thereafter, a portion of the second nail portion can be removed.

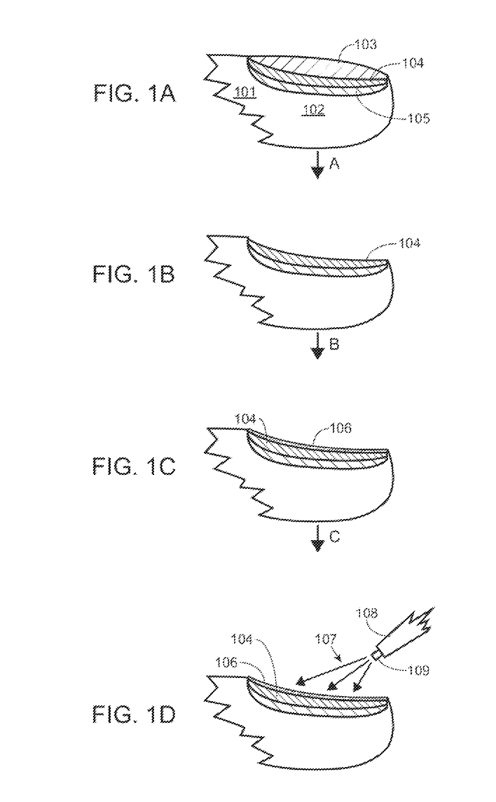

FIGS. 1A-1D provide diagrams representing a method of treating an infection according to embodiments of the present disclosure. These diagrams provide schematic partial cross-sections of an appendage 101 of a subject, such as a mammal. Such an appendage includes one or more supporting tissues 102 connected to a nail composed of a first nail portion 103, such as a nail plate, a second nail portion 104, e.g., an epidermis portion, and a third nail portion 105, such as a dermis portion. The second and third nail portions together constitute a nail bed portion. The nail bed is located under the nail plate. Also, the nail plate is composed of keratin.

An infection, such as a fungal infection can be an infection of the nail bed, such as the second nail portion 104, e.g., an epidermis portion, and a third nail portion 105, such as a dermis portion. As such, each of these layers or one of these layers, such as the second nail portion 104, can include one or more infection-causing aspects, such as a fungus. An infection can also grow in to the first nail portion 103, for example the nail plate, thereby destroying its structure and/or changing its color.

As noted above, aspects of the methods include removing, as shown schematically by arrow “A,” a first nail portion 103 of an appendage a mammal including the dermal fungal infection to expose a second nail portion 104 of the appendage. Embodiments of the methods also include applying a laser absorbing compound 106 to the second nail portion 106 of the appendage. Applying the laser absorbing compound 106 to the second nail portion 106 is shown schematically by arrow “B.” The methods also include irradiating, such as irradiating with laser radiation 107, emitted from a laser 108 having a lens 109, the second nail portion 104 and/or the laser absorbing compound 106 with a laser 107 of a treatment system to selectively heat the laser absorbing compound 106 and thereby denature tissue, such as tissue of a second nail portion, and/or dermal fungus associated with the dermal fungal infection and/or present in the second nail portion 104. The methods may thereafter include removing a portion, such as a top layer portion opposite a bottom layer portion, of the second nail portion 104 and/or the laser absorbing compound 106. Any one or combination of the steps referred to above can then optionally be repeated again. An anti-infectious compound, such as an anti-fungal agent, may then be applied to the second nail portion.

Removing a portion of the second nail portion can include contacting the portion of the second nail portion with a tissue removing device and/or cutting or grinding off the nail portion with the tissue removing device. Also, in some versions, the methods can further include inputting a set of instructions to a control unit of the treatment system operatively connected to a tissue removing device, wherein upon receiving the set of instructions, the control unit moves the tissue removing device with respect to the appendage to remove the portion of the second nail portion. The control unit can regulate the amount of the second portion removed by controlling the depth and/or duration of grinding or cutting.

As noted above, the subject disclosure includes methods of treating an infection in an appendage of a mammal. The terms “treating” and “treatment”, as used herein, refer to taking one or more actions, such as following one or more of the protocols described herein, to alleviate the ailment and/or symptoms of the ailment. Treatment can include fully or partially killing one or more sources of the infection, such as a fungus.

Symptoms of ailments, such as infections, disclosed herein may include but are not limited to discomfort, such as pain and/or tingling and/or aching, and/or burning, and/or numbness and/or hypersensitivity and/or weakness, and/or stiffness, and/or atrophy, and/or circulatory changes, in a part of a subjects body, for example, a nail, or discolored, or deformed, such as cracked or otherwise weakened nails and treatment may include alleviating or eliminating any one or combination of such symptoms. Taking one or more actions to treat a subject may include not taking action at a particular juncture in a treatment protocol or not taking immediate action to treat the ailment. The subject methods also include substantially alleviating an ailment, such as an infection, and/or symptoms of the ailment, in a subject permanently, e.g., for the remaining life of the subject, or temporarily, such as for a period of time of 5 minutes or more, such as one hour or more, such as one day or more, such as one month or more, such as one year or more, such as five years or more.

Infected appendages according to the subject embodiments can include a finger and/or a toe. The dermal infection can include an infection of a nail portion of the appendage and can be a fungal infection. Examples of fungal infections which can be treated according to the subject embodiments include, but are not limited to onychomycosis and tinea capitis. Onychomycosis infections can be caused by species of any of the following types: Candida, and/or Scopulariopsis breyicaulis. Infection-causing nail pathogens according to various aspects are dermatophytes Trichophyton rubrum and/or Trichophyton mentagrophytes.

Infections according to the embodiments can also include cutaneous dermatophytoses and may be of any of the following types: Tinea cruris, tinea corporis, tinea imbricate, tinea pedis. Infections can also be caused by species of any of the following types: Aspergillus fumigatus, candida glabrata, candida tropicalis, candida kreusi, fusarium. Dermal infections as provided herein can be caused by species of any of the following types: trichophyton, microsporum epidermophyton, dermatiaceae, filamentous fungi, or yeast. Infections according to the embodiments can also include non-dermatophyte infections. Infections according to the subject embodiments can also include secondary infections, such as bacterial infections caused by a primary infection, such as onychomycosis.

The methods can include removing or substantially removing a first nail portion of an appendage a mammal including the dermal fungal infection to expose a second nail portion of the appendage. As used herein, “substantially” means to a great or significant extent, such as almost fully or almost entirely. In various embodiments, the infection and the aspect causing the infection, such as the fungus, is located within or substantially within the second nail portion, or the second nail portion and the first nail portion. In some aspects, removing a nail portion, such as a first and/or second nail portion, includes contacting a nail portion, such as a first and/or second nail portion, with a tissue removing device. The tissue removing device can be employed to cut or grind one or more portions from the first nail portion until the first nail portion is removed or substantially removed from the appendage. The tissue removing device can also be employed to cut or grind one or more portions from the second nail portion after the nail portion is irradiated. The subject systems can also include a tissue removing device operatively coupled to the control unit such that the control unit moves the tissue removing device with respect to the appendage to remove a nail portion of the infected portion of the mammal.

According to the disclosed aspects, a tissue removing device can be a grinder, such as device including a grinding wheel, such as a diamond-coated grinding wheel. A grinding wheel can be configured to rotate while contacting and thereby grinding off tissue portions. A tissue removing device can also be a tissue cutting device such as a surgical tissue cutter, such as a device having a cutting laser or one or more knife.

After the first nail portion is removed, the methods can also include applying a radiation absorbing compound, such as a laser absorbing compound, to a second nail portion of the appendage. Applying such a compound may include spraying and/or spreading a layer thereon with an applicator. A radiation absorbing, such as laser absorbing, compound or absorption compound, as referred to herein is a compound which absorbs radiation, for example, laser radiation, significantly more readily than tissue. A laser absorbing compound can have a peak spectral absorption of 1000 nm or less, such as 850 nm or less, such as 830 nm or less, such as 815 nm or less, such as 810 nm or less, such as 805 nm or less, such as 800 nm or less. A laser-absorbing compound can have a peak spectral absorption in an inclusive range of, for example 600 nm to 1000 nm, such as 700 nm to 900 nm, such as 750 nm to 850 nm, such as 790 nm to 810 nm. By “inclusive”, as used herein in association with ranges means that the range includes both of the provided endpoints. All ranges provided herein are inclusive, regardless of whether the term is provided in association with a particular range or unless indicated otherwise. A laser-absorbing compound can have a peak spectral absorption in the near infrared region. A laser-absorbing compound can also emit fluorescence in an inclusive range of 750 nm and 950 nm, such as 750 nm to 850 nm such as 790 nm to 810 nm.

Irradiation of a laser absorbing compound and the corresponding absorption can in turn result in the compound being heated, such as being heated to 80° C. or more, such as being heated from 20° C. to 80° C. or more. Such heating can include heating the compound and/or tissues contacting and/or adjacent to the compound, such as a second nail portion, 10° C. or more, 20° C. or more, 30° C. or more, 40° C. or more, 50° C. or more, 60° C. or more, 70° C. or more, or 80° C. or more. Irradiation of a laser absorbing compound and the corresponding absorption can in turn result in tissues, e.g., a second nail portion, such as an infected second nail portion, adjacent to or contacting the laser absorbing compound being heated, any of the amounts provided above, and may include heating the tissues to 80° C. or more.

Examples of laser absorbing compounds include dyes, such as cyanine dyes including indocyanine green (ICG), also known as sodium 4-[2-[(1E,3E,5E,7Z)-7-[1,1-dimethyl-3-(4-sulfonatobutyl)benzo[e]indol-2-ylidene]hepta-1,3,5-trienyl]-1,1-dimethylbenzo[e]indol-3-ium-3-yl]butane-1-sulfonate and having the molecular formula C₄₃H₄₇N₂NaO₆S₂ and a molecular weight of 774.962849 g/mol. ICG also has a chemical formula as follows:

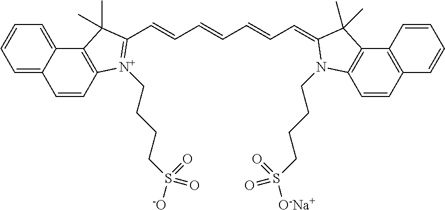

Laser absorbing compounds such as ICG can be a powder in the form of small crystals. The powder, as applied according to the subject methods, can be dissolved in a solvent before applying the laser absorbing compound to the nail portion. A laser absorbing compound can have solvability in many solvents.

Further examples of laser absorbing compounds include IR Dye® 800CW and Alexa Fluor 790®. Alexa Fluor 790® has a molecular weight at or around 1750 g/mol. Also, IRDye® 800CW has the molecular formula C₄₉H₅₄N₃Na₃O₁₄S₂₄ and a molecular weight of 1106.19 g/mol. IRDye® 800CW, for example, also has a chemical formula as follows:

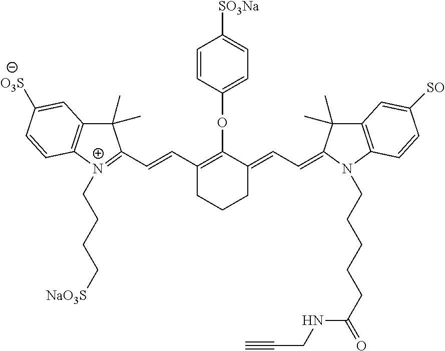

Laser absorbing compounds can also include one or more solvent, such as water and/or dimethyl sulfoxide (DMSO). Exemplary other solvents which can be included are Ethanol, hexane, tetrachloroethylene, toluene, methyl acetate, ethyl acetate, acetone, acetonitrile, tBuOH, benzene, carbon tetrachloride, chloroform; cyclohexane, ether, diisopropyl ethylamine, ethyl acetate, hexage, pentane, pyridine, and/or toluene, or any combination thereof.

After the laser absorbing compound is applied, the second nail portion can be irradiated according to the subject methods with a laser of a treatment system. Such irradiation can include selectively heating the laser absorbing compound, such as heating the compound to 80° C. or more. Such heating can include killing an infection-causing aspect, such as a dermal fungus. Such heating can also include killing tissue, such as tissue of the second nail portion so that it can easily be removed, such as be removed while minimizing irritation and/or discomfort. The phrase “selectively heating” as used herein refers to heating a particular aspect, e.g., a compound and/or a tissue having a compound applied directly thereon, while not heating adjacent aspects, such as tissues, by the same amount. For example, selectively heating an aspect can include heating the aspect to a first temperature while not heating an adjacent aspect or heating the adjacent aspect to a second temperature which is 80° C. or less, 50° C. or less, 40° C. or less, 30° C. or less, 20° C. or less, 15° C. or less, 10° C. or less, 5° C. or less, 3° C. or less, or 1° C. or less than the first temperature. Also, for example, selectively heating an aspect can include heating the aspect to a first temperature, such as a temperature at which an infection-causing aspect such as a fungus will die, for example, a temperature of 80° C. or more, while not heating an adjacent aspect, such as a tissue, or heating the adjacent aspect to a second temperature which is less than the first temperature and at which the adjacent aspect, such as tissue, will not die or not substantially die. Selectively heating an aspect can also include heating the aspect to a first temperature, such as a temperature at which tissue, such as tissue of the second layer, will denature, such as 40° C. or more, while not substantially heating an adjacent aspect, e.g., a tissue beneath and/or adjacent to the second layer.

In addition, selectively heating an aspect can include heating the aspect to a first temperature, such as a temperature at which an infection-causing aspect such as a fungus will die, such as a temperature of 80° C. or more, while heating a first adjacent aspect, e.g., an infected tissue, contacting the initial aspect, such as the radiation absorbing compound, to a same or substantially same temperature. Such a protocol can also include not heating a second adjacent aspect, such as a second tissue, such as a supporting tissue or another tissue layer contacting the first aspect but not contacting the radiation-absorbing compound, or heating the second adjacent aspect to a second temperature which is less than the first temperature and at which the second adjacent aspect, for example, tissue, will not die or not substantially die.

Selectively heating the laser absorbing compound can also include maintaining the laser absorbing compound at a temperature, such as a temperature of 80° C. or more for a period of time, e.g., 1 sec or more, 30 sec or more, 1 min or more, 3 min or more, 5 min or more, 10 min or more, or 20 min or more, or 1 hour or more, or a time sufficient to kill or substantially kill an infection-causing aspect such as a fungus and/or a tissue, such as a second layer, having the laser absorbing compound applied directly thereon.

Any of the method steps provided herein can also include applying to the subject appendage an anesthetic agent, also referred to as an anesthetic, such as a local anesthetic, and/or an anti-infectious compound before and/or after each step is performed. For example, the methods can include applying to the second nail portion an anesthetic and/or an anti-infectious compound after the first nail portion is removed and/or after the second nail portion is irradiated and/or partially removed. Application of such an anesthetic may reduce potential pain experienced by the subject undergoing the procedure. Application of such an anti-infectious compound may kill bacteria, fungus or other infection-causing agents. The anesthetic can be applied to the first and/or second nail portions and/or supporting or other appendage tissue. Examples of anesthetic agents which can be treated according to the subject embodiments include, but are not limited to: lidocaine, acetamide, 2-(diethylamino)-N-(2,6-dimethylphenyl); prilocaine, N-(2-methylphenyl)-2-(propylamino); benzocaine; butamben; dibucaine; oxybuprocaine; pramoxine; proparacaine; proxymetacaine; tetracaine and/or eutectic mixtures such as: Eutectic Mixture of Local Anesthetics (EMLA®) (including Lidocaine®, and Prilocaine®).

Furthermore, examples of anti-infectious compounds which can be treated according to the subject embodiments include, but are not limited to: imidazole antifungal drugs such as luliconazole, (2E)-[(4R)-4-(2,4-Dichlorophenyl)-1,3-dithiolan-2-ylidene](1H-imidazol-1-yl)acetonitrile; econazole, (RS)-1-{2-[(4-Chlorophenyl)methoxy]-2-(2,4-dichlorophenyl)ethyl}-1H-imidazole; terbinafine, [(2E)-6,6-dimethylhept-2-en-4-yn-1-yl](methyl)(naphthalen-1-ylmethyl)amine, griseofulvin, fluconazole, itraconazole, or other effective skin anti-fungus compounds, such as solutions/creams, with a high skin penetration.

Following irradiation, a tissue removing device can also be employed to removing, such as by cutting or grinding one or more portions from the second nail portion. Such steps of irradiation and grinding can be successively repeated until an infection is eradicated.

As such, in various aspects, the methods include repeating, e.g., repeating one or more times, e.g., two or more, five or more or ten or more or ten or fewer, five or fewer, or two or fewer, steps of applying a laser absorbing compound to the second nail portion of the appendage, irradiating the second nail portion with a laser of a treatment system to selectively heat the laser absorbing compound, and/or removing a portion, e.g., a layer such as a top layer, of the second nail portion of the appendage.

The steps can be repeated at a frequency of once every four weeks or less, once every two weeks or less, once per week or less, or once per day or less. In some aspects, the frequency is increased as a subject progressively develops a higher pain tolerance in response to successive treatments. In some versions, the methods further include repeatedly administering an anti-infectious compound to the second nail portion of the appendage at a frequency of once per day or more. The methods can also include increasing an amount of laser radiation delivered each time the second nail portion is irradiated with the laser. In some aspects, the amount of laser radiation delivered is increased as a subject progressively develops a higher pain tolerance in response to successive treatments. Accordingly, a greater amount of the second nail portion can be removed with each successive repetition of the treatment steps.

Also, the terms “tissue”, or “tissues” as used herein, refer to one or more aggregates of cells in a subject (e.g., a living organism, such as a mammal, such as a human) that have a similar function and structure, or to a plurality of different types of such aggregates. Tissue may include, for example, organ tissue, muscle tissue, such as skeletal muscle, connective tissue, nervous tissue and/or epithelial tissue. One or more tissues which are functionally grouped together may form an organ. Tissues may also be one or more soft tissues. “Soft tissue”, as used herein, refers to tissue that connects, supports, or surrounds other structures, for example, organs, of a subject body and which is not bone. Soft tissues may be subcutaneous and may include connective tissue, such as tendons, fascia, ligaments, skin, fibrous tissues, fat and/or synovial membranes, or any combinations thereof, as well as tissues other than connective tissues, including muscles, nerves, and/or blood vessels, or any combinations thereof.

As noted above, in some versions, the methods are performed on a subject. In various instances, a subject is a “mammal” or a “mammalian” subject, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the subject is a human. The term “humans” can include human subjects of both genders and at any stage of development (e.g., fetal, neonates, infant, juvenile, adolescent, and adult), where in certain embodiments the human subject is a juvenile, adolescent or adult. While the systems and methods described herein can be applied in association with a human subject, it is to be understood that the subject devices and methods can also be applied in association with other subjects, that is, on “non-human subjects.”

In some versions, the methods include stably associating an appendage of a mammal, such as a hand and/or foot and/or finger and/or toe, with an alignment stage of the treatment system. By “stably associating” and/or “stably associated” is meant placing in a contacting orientation, such as a contacting orientation wherein the surface area of the touching portions are maximized, and/or affixing to, such as affixing to with a mild adhesive, such that the disclosed methods, systems, and components thereof may be effectively employed.

The methods also include moving at least a portion of the alignment stage, such as a first or second portion, and/or the laser, such as a lens, to align in treatment proximity the second nail portion and the laser. By “treatment proximity” as used herein, is meant an orientation in which an infected portion of a mammal and/or a substance thereon can be irradiated by radiation in accordance with the subject methods to effectively treat the infection, such as by killing an infection-causing aspect such as a fungus. Aligning an aspect in treatment proximity can include positioning the portion of the mammal an effective distance, as described herein, away from the laser lens by moving the infected portion toward and/or away from the lens along an irradiation axis and/or the lens toward and/or away from the infected portion along the irradiation axis.

The methods also include, in various aspects, aligning in treatment proximity the second nail portion and the laser using radiation emitted by the laser as a visual alignment guide. In such embodiments, radiation at a first wavelength which can be optically detected by an un-assisted human eye is used to position the laser lens with respect to one or more portions of the alignment stage. The radiation is used to produce an alignment signal, such as the convergence of two lines or a degree of focus of one or more beam of the radiation, which is optically detected by the user. Based on the signal, the user can make an input to the system or manually adjust one or more aspects of the system to align the aspects in treatment proximity.

In various aspects, the methods include obtaining infection image data with an imaging unit. In some aspects, obtaining such image data includes taking one or more photo of a portion of a mammal, such as an infected portion of a mammal. Also, in some aspects, the methods include comparing the infection image data with infection image data stored in a database. Such a comparison can be performed automatically with a processor. The methods also can include storing the infection image data, such as one or more photos and/or videos, in a database. Also, in some versions, determining one or more characteristics of a sample includes performing infection image analysis on image data, such as by comparing the image data with data stored in a library of image data and/or associated characteristics, to for example produce an irradiation pattern output with the control unit.

According to aspects of the disclosed methods, the laser and the alignment stage are moved relative to one another along an irradiating pattern to irradiate the infected portion of the mammal. When a portion of a mammal is irradiated, radiation, such as laser radiation, is emitted thereon and/or onto a substance on the mammal portion such as a laser-absorbing compound. Performing such irradiation can include killing one or more causes of the infection such as a fungus. Also, movement between a laser and an alignment stage can be performed by moving one of the elements with respect to the other in an irradiating pattern and can be driven by one or more, such as a plurality, such as two, movement units. The irradiating pattern can be specific and pre-selected. As such, the methods include pre-selecting an irradiating pattern and entering an input corresponding with the selected patter into the control unit. An irradiating pattern can be a linear and/or curved pattern and can be performed by moving the alignment stage and/or the laser lens in a curved motion and/or a linear motion with respect to one another. Accordingly, the subject methods include moving the alignment stage and/or the laser lens in a curved motion and/or a linear motion with respect to one another along the patterns illustrated schematically in FIGS. 4 and 5.

In various embodiments, the methods include scanning an area of an infected portion of a mammal by moving the laser or apportion thereof with respect to the infected portion and/or an alignment stage or a portion thereof at a speed of at 2.5 cm/s or less, 2.0 cm/s or less, such as 1.5 cm/s or less, such as 0.5 cm/s or less, such as 0.3 cm/s or less, such as 0.1 cm/s or less. Such a speed can also be 2.5 cm/s or more, 2.0 cm/s or more, 1.5 cm/s or more, 0.5 cm/s or more, 0.3 cm/s or more, or 0.1 cm/s or more. Such a speed may range, for example, from 0.1 cm/s to 5 cm/s, such as 0.5 to 2.5 cm/s, such as 1.0 to 2 cm/s. The methods can include irradiating the infected portion of a mammal at such a speed along a scanning line, which is a continuous linear pattern of irradiation or a linear pattern of irradiation areas, such as a pattern forming a single line. The methods include irradiating scanning lines successively until an entire infected portion of a mammal is irradiated.

A scanning line can be a distance away from another scanning line of 500 μm or less, such as 400 μm or less, such as 300 μm or less, such as 200 μm or less, such as 150 μm or less, such as 100 μm or less, such as 50 μm or less, such as 10 μm or less, such as 1 μm or less. A scanning line can also be a distance away from another scanning line of 500 μm or more, 400 μm or more, 300 μm or more, 200 μm or more, 150 μm or more, 100 μm or more, 50 μm or more, 10 μm or more. A scanning line can also be a distance away from another scanning line wherein the distance is in a range of 1 μm to 500 μm, such as 100 μm to 300 μm, such as 150 μm to 250 μm. Also, according to the subject embodiments, an irradiation area can be a distance away from another adjacent irradiation area which is equal to any of the distances between scanning lines provided herein.

In some versions, irradiating, such as irradiating a second nail portion, includes continuously or periodically irradiating the second nail portion with radiation, while continuously moving the second nail portion and/or a portion of an alignment stage with which the appendage including the second nail portion is stably associated, relative to the laser or a portion thereof, such as a lens. In some versions, irradiating, such as irradiating a second nail portion, includes continuously or periodically irradiating the second nail portion with radiation, such as laser radiation, while periodically moving the second nail portion and/or a portion of an alignment stage with which the appendage including the second nail portion is stably associated, relative to the laser or a portion thereof, such as a lens.

In some aspects, the methods include not moving the second nail portion and/or a portion of an alignment stage with which the appendage including the second nail portion is stably associated, relative to the laser or a portion thereof, such as a lens, while irradiation is performed. In some aspects, the methods include only moving the second nail portion and/or a portion of an alignment stage with which the appendage including the second nail portion is stably associated, relative to the laser or a portion thereof, such as a lens, while irradiation is performed. As such, in some aspects of the methods, irradiating the second nail portion includes periodically irradiating the second nail portion with the laser and periodically moving the second nail portion relative to the laser.

Furthermore, in various aspects, the methods include inputting a set of instructions to a control unit of the treatment system. The control unit can be configured such that wherein upon receiving the set of instructions, the control unit moves or emits an output causing movement of the second nail portion in a continuous irradiation pattern and/or a periodic irradiation pattern with respect to the laser.

### Kits

Also provided are kits that at least include the subject systems or system components, such as devices, and which may be used according to the subject methods. The disclosed kits, in various embodiments, include any of the embodiments of the devices described herein or any combinations thereof. The subject kits may include one or more, such as a plurality, e.g., two or more, of the system components as described herein. The components of the subject kits may also include media content, such as publications and/or software, describing, illustrating and/or relating to one or more embodiments of the methods described herein.

In various embodiments, the kits include a treatment system including a laser and/or an alignment stage. The alignment stage can be configured for receiving an infected portion of a mammal including the dermal fungal infection and aligning the infected portion of the mammal with the laser. Also, the laser and the alignment stage are, in some aspects, operatively connected and configured to move relative to one another along an irradiating pattern to irradiate the infected portion of the mammal. The kits can also include a radiation absorbing compound, such as a laser absorbing compound, such as ICG. Such a compound can include a solvent, such as DMSO. Radiation absorbing compounds according to embodiments of the subject kits can also have a peak spectral absorption of 850 nm or less, such as 820 nm or less, such as 810 nm or less, such as 800 nm or less.

The subject kits can also include a tissue removing device for removing a first nail portion of the infected portion of the mammal. The tissue removing device can be operatively coupled to the control unit so that the control unit controls one or more aspects of the removal, such as the duration and/or depth, of a nail portion, e.g., a second nail portion, of the infected portion of the mammal. In various aspects, the kits include a display element for displaying one or more aspects of the elements and/or systems described herein. Also, in some variations, kits include a power source for the systems described herein.

In various embodiments, the kits which are disclosed herein include instructions, such as instructions for using the devices and/or performing the subject methods. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging etc.). In other embodiments, the instructions are present as an electronic storage data file present on a suitable computer readable storage medium, such as a Portable Flash drive, CD-ROM, diskette, etc. The instructions may take any form, including complete instructions for how to apply the methods and/or devices or as a website address with which instructions posted on the world wide web may be accessed.

In addition, embodiments of the disclosed kits or their components may be used according to any of the embodiments of the methods described herein or combinations thereof.

### Utility

As demonstrated above, the subject systems and methods are directed to treating dermal infection in an appendage of a subject such as a mammal. As such, the subject methods are suitable for use in a variety of different applications, where particular applications include addressing abnormal conditions, such as infections may be desirable. Particular applications in which the subject systems and methods find use include the alleviation or treatment of onychomycosis infections.

Furthermore, although both topical and oral systemic antifungal agents are available, they are not always effective, carry medical risks, are associated with potentially significant drug-drug interactions, and may be unacceptable to a patient and healthcare provider alike. Systemic antifungal drugs are a mainstay of therapy for treating fungal infection. When taken for a 12-week course, oral terbinafine has a mycologic cure rate of 71%-82% and a maximal clinical response rate of 60%-70%. However, patients are often poorly compliant with the lengthy duration of treatment, resulting in sub-therapeutic concentrations reaching the nail plate. Furthermore, antifungal drugs such as terbinafine can elevate liver enzymes and has progressed to fulminant liver failure. As such, it requires routine blood testing and is contraindicated in patients with chronic or active liver disease. Additional related events can include headache, loss of taste, and abdominal discomfort and induction of a lupus-like syndrome. Itraconazole, another systemic antifungal drug utilized to manage onychomycosis, has much lower mycologic and clinical clearing rates, and may induce cardiac toxicity; it is also associated with a plethora of drug-drug interactions.

Treating an infection in a subject according to the disclosed subject matter may increase the lifespan of the subject and/or assist the subject in being comfortable. Treating an infection in a subject according to the subject methods and with the subject systems may also reduce the health risk to the subject and/or discomfort associated with an alternative treatment, such as a surgical procedure and/or an enteral drug as described above. A combination of topical and surgical treatment of infection as described herein provides not only a high success rate in alleviating an infection but is also almost painless, and can be conducted in a reasonably short treatment time.

### Example

The following example is put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### I. Example 1

An infected, mostly dead nail plate was removed. It was easily grounded out without any pain. Thereafter, Indocyanine green (ICG) solution in Dimethyl sulfoxide (DMSO) having a concentration of less than 1 mg/ml was applied.

The nail bed was then dried out and then EMLA® (including Lidocaine®, acetamide, 2-(diethylamino)-N-(2,6-dimethylphenyl); and Prilocaine®, N-(2-methylphenyl)-2-(propylamino)) deep skin numbing cream was applied as a local anesthetic.

A time period of 30 minutes was then taken without further action to allow the anesthetic to numb the relevant treatment area.

Laser radiation was applied through a scanning device over overlapping nail area with a rectangle scanning area. Laser wavelength is 800 nm where ICG has an absorption peak. As a result, significant absorption occurred only at the area where ICG was applied. The laser radiation was, and should be, adjusted to the scanning system. Also, two step-motor stages were combined together as an alignment stage to provide a 2-D scanning system.

Scanning was performed with a reasonable scanning speed of 1 inch per second with another parallel scanning line 200 micrometers apart. 200 micrometers is a trade-off size because most multi-mode lasers can be coupled with fiber with typical output diameter 200 micrometers on the one hand. Also, typical heat dissipation time from 200×200 microns area in skin is 40 milliseconds (L̂2/4/A) where L is diameter of the heating area 200 microns (2×10̂-4 m), and A is water diffusivity (1.4×10̂-7 m̂2/s). Therefore the dissipation time=(2×10̂-4 m) ̂2/4/(1.4×10̂-7 m̂2/s)˜70 ms and pulse duration is around 10 milliseconds. As such, a scanning speed of 200 microns per 10 milliseconds or 2 cm/s˜1 inch/s is provided.

The typical power of the laser radiation required to heat water, such as water within an appendage of a mammal, from 20° C. (room temperature) to 80° C. (minimum temperature when funguses start dying) was also calculated as follows: Water heat capacity is H=4.1855 J/g/K, density is D=1 g/cm̂3, therefore required energy for volume cm̂3 is E=H*D*(80−20)=250 J. For area with smaller dimensions 1 mm by 1 mm and depth 1 mm or 1 mm̂3=10̂-3 cm̂3 then E=250 J*10̂-3=250 mJ and for 200 microns×200 microns×1 mm microns 250 mJ/25=10 mJ.

As the above estimation of the pulse duration 10 ms the CW power of the laser should be P=10 mJ/10 ms=1 W, which is not a problem for a typical diode CW laser at 800 nm. One such example is the QSP-808-4 offered by QPhotonics® with P=4 W.

Laser treatment can be painful at the full power at the first few scans, however the human adaptively to the pain level changes. If the laser power at the first scan produced a moderate level of pain, the same level of power for the second scan produced small or negligible level of pain. Therefore for the initial scan, the laser power was adjusted to the level of the pain tolerance for the individual patient and then steadily was increased for each successive scan until the treatment power was reached. As such, in some aspects, the methods include increasing an amount of laser radiation delivered in a scan periodically, such as each time the second nail portion is irradiated with the laser. Increasing the amount of laser radiation delivered can increase the efficiency of scans because more selective heating can be achieved with more radiation. The methods also include adjusting the amount of laser radiation delivered in a scan based on the pain tolerance of a subject. For example, when pain tolerance increases, the amount of laser radiation delivered can be increased.

After the laser treatment, and when the anesthetic was still in effect, the dead skin was carefully ground out and anti-fungus compounds were applied. Suitable anti-fungus compounds include Luliconazole, Econazole, Terbinafine, or any other effective skin anti-funguses compound solutions/creams having a high skin penetration.

The procedure was repeatedly performed once a week until the infection was fully cured.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A system for treating a dermal fungal infection in a mammal, the system comprising:
a laser;
an imaging unit for obtaining infection image data;
an alignment stage for receiving an infected portion of a mammal comprising the dermal fungal infection and aligning the infected portion of the mammal with the laser; and
a control unit for controlling relative movement between the laser and the alignment stage along an irradiating pattern and irradiating the infected portion of the mammal based on the infection image data.

2. The system according to claim 1, wherein the laser comprises a diode configured to emit radiation having a wavelength of 850 nm or less.

3. The system according to claim 2, wherein the laser comprises a diode configured to emit radiation having a wavelength of 810 nm or less.

4. The system according to claim 1, wherein the laser comprises a first diode configured to emit radiation having a wavelength of 850 nm or less and a second diode configured to emit radiation detectable by an un-assisted human eye.

5. The system according to claim 1, wherein the irradiating pattern is a continuous irradiating pattern.

6. The system according to claim 1, wherein the irradiating pattern is a periodic irradiating pattern.

7. The system according to claim 1, wherein the system further comprises a movement unit operatively connected to the laser and configured for moving at least a portion of the laser.

8. The system according to claim 1, wherein the system further comprises a movement unit operatively connected to the alignment stage and configured for moving at least a portion of the alignment stage.

9. The system according to claim 8, wherein the movement unit is configured for moving the infected portion of the mammal in a first dimension and a second dimension perpendicular to the first dimension.

10. The system according to claim 1, wherein the laser comprises a diode, a cooler, a lens, an optical fiber, or any combination thereof.

11. The system according to claim 10, wherein the laser comprises a lens having a cross-sectional diameter of 2.5 cm or less.

12. The system according to claim 1, further comprising a power source operatively connected to the laser, the alignment stage, or both the laser and the alignment stage.

13. A method of treating a dermal fungal infection in an appendage of a mammal, the method comprising:
removing a first nail portion of an appendage of a mammal comprising the dermal fungal infection to expose a second nail portion of the appendage;
applying a laser absorbing compound to the second nail portion of the appendage; and
irradiating the second nail portion with a laser of a treatment system to selectively heat the laser absorbing compound.

14. The method according to claim 13, wherein the laser absorbing compound has a peak spectral absorption of 850 nm or less.

15. The method according to claim 14, wherein the laser absorbing compound has a peak spectral absorption of 810 nm or less.

16. The method according to claim 13, wherein the laser absorbing compound comprises indocyanine green.

17. The method according to claim 16, wherein the laser absorbing compound further comprises a solvent.

18. The method according to claim 17, wherein the solvent comprises dimethyl sulfoxide.

19. The method according to claim 13, wherein the laser absorbing compound is selectively heated to 80° C. or more.

20. The method according to claim 13, further comprising stably associating the appendage of the mammal with an alignment stage of the treatment system.

21. The method according to claim 20, further comprising moving at least a portion of the alignment stage to align in treatment proximity the second nail portion and the laser.

22. The method according to claim 13, further comprising aligning in treatment proximity the second nail portion and the laser using radiation emitted by the laser as a visual alignment guide.

23. The method according to claim 13, wherein irradiating the second nail portion comprises continuously irradiating the second nail portion with the laser while continuously moving the second nail portion relative to the laser.

24. The method according to claim 13, wherein irradiating the second nail portion comprises periodically irradiating the second nail portion with the laser while continuously moving the second nail portion relative to the laser.

25. The method according to claim 13, wherein irradiating the second nail portion comprises periodically irradiating the second nail portion with the laser and periodically moving the second nail portion relative to the laser.

26. The method according to claim 13, wherein removing a first nail portion comprises contacting the first nail portion with a tissue removing device.

27. The method according to claim 13, further comprising administering an anesthetic compound to the second nail portion of the appendage.

28. The method according to claim 13, further comprising removing a portion of the second nail portion of the appendage.

29. The method according to claim 28, further comprising administering an anti-infectious compound to the second nail portion of the appendage.

30. The method according to claim 13, further comprising inputting a set of instructions to a control unit of the treatment system, wherein upon receiving the set of instructions, the control unit moves the second nail portion in a continuous irradiation pattern or a periodic irradiation pattern with respect to the laser.

31. The method according to claim 13, wherein the dermal fungal infection is onychomycosis.

32. The method according to claim 13, wherein the appendage is a finger or toe.

33. A kit for treating a dermal fungal infection in a mammal, the kit comprising:
a treatment system comprising:
a laser;
an imaging unit for obtaining infection image data;
an alignment stage for receiving an infected portion of a mammal comprising the dermal fungal infection and aligning the infected portion of the mammal with the laser, and
a control unit for controlling relative movement between the laser and the alignment stage along an irradiating pattern and irradiating the infected portion of the mammal based on the infection image data; and
a laser absorbing compound.

34. The kit according to claim 33, wherein the laser absorbing compound comprises indocyanine green.

35. The kit according to claim 34, wherein the laser absorbing compound further comprises a solvent.

36. The kit according to claim 35, wherein the solvent comprises dimethyl sulfoxide.

37. The kit according to claim 33, wherein the laser absorbing compound has a peak spectral absorption of 850 nm or less.

38. The kit according to claim 33, further comprising a tissue removing device for removing a nail portion of the infected portion of the mammal.

39. The method according to claim 28, further comprising repeating steps of applying a laser absorbing compound to the second nail portion of the appendage, irradiating the second nail portion with a laser of a treatment system to selectively heat the laser absorbing compound, and removing a portion of the second nail portion of the appendage.

40. The method according to claim 39, wherein the steps are repeated at a frequency of once per week or less.

41. The method according to claim 39, further comprising repeatedly administering an anti-infectious compound to the second nail portion of the appendage at a frequency of once per day or more.

42. The method according to claim 39, further comprising increasing an amount of laser radiation delivered each time the second nail portion is irradiated with the laser.

43. The method according to claim 1, wherein the infection image data comprises luminescence detected from the infection.

44. The kit according to claim 38, wherein the tissue removing device is operatively coupled to the control unit so that the control unit controls the removal of the nail portion of the infected portion of the mammal.

45. The system according to claim 1, further comprising a tissue removing device operatively coupled to the control unit such that the control unit moves the tissue removing device with respect to the appendage to remove a nail portion of the infected portion of the mammal.

46. The method according to claim 28, wherein removing a portion of the second nail portion comprises contacting the portion of the second nail portion with a tissue removing device.

47. The method according to claim 28, further comprising inputting a set of instructions to a control unit of the treatment system operatively connected to a tissue removing device, wherein upon receiving the set of instructions, the control unit moves the tissue removing device with respect to the appendage to remove the portion of the second nail portion.
